(19) United States
(12) Patent Application Publication
(10) Pub. No.: US 2009/0163877 A1
(43) Pub. Date: Jun. 25, 2009

(54) Cannula For An Injection Device, The Cannula Having A Tapered End, And A Method For Manufacturing the Same

(75) Inventors: Lasse Wengel Christoffersen, Soborg (DK); Henrik Sonderskov Klint, Lyngby (DK); Karl Brian Nielsen, Kastrup (DK); Michael Andersen, Malmö (SE)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordosk A/S, Bagsvaerd (DK)

(21) Appl. No.: 12/298,266

(22) PCT Filed: Apr. 25, 2007
(86) PCT No.: PCT/EP07/54072

§ 371 Date: Feb. 23, 2009

(30) Foreign Application Priority Data
Apr. 26, 2006 (EP) 06008651.9

(51) Int. Cl. A61M 5/32; A61M 5/00; B21D 22/20

(52) U.S. Cl. 604/240; 604/272; 72/343; 72/361; 72/372

(57) ABSTRACT

A cannula (100) for an injection device, the cannula defining a tapered end part termination (102) in a distal end for insertion into the tissue of a living being, wherein the hardness of the cannula decreases in the direction of the distal end. A tool (104) for providing a tapered portion of a cannula, the tool defining a tapered cavity (106). A method of manufacturing a cannula defining a tapered end part, by forcing a distal end of an tubular element into a tapered cavity of a tool.

### FIELD OF THE INVENTION

The present invention relates to a cannula for an injection device, the cannula defining a tapered end part termination in a distal end for insertion into the tissue of a living being. Especially, the present invention relates to a cannula wherein the hardness of the cannula decreases in the direction of the distal end. Moreover, the present invention relates to a tool for providing a tapered portion of a cannula, the tool defining a tapered cavity. Furthermore, the present invention relates to a method of manufacturing a cannula defining a tapered end part, by forcing a distal end of an tubular element into a tapered cavity of a tool.

### BACKGROUND OF THE INVENTION

The pain experienced by a patient when introducing a cannula into the patient's tissue is highly dependent on the diameter and the shape of the cannula. Conventional cannula's have relative large diameters, and, thus, cause an undesired high level of pain. In order to overcome this problem, new designs for cannula's have been introduced, wherein the diameter of the tissue-penetrating part of the cannula is reduced.

One such example is known from EP 1 188 456 A1, which discloses a drug injection needle comprising a puncturing needle part and a drug introducing needle part capable of communicating with the inside of a drug container. The drug introducing needle part has a larger outer diameter than that of the puncturing needle part. An intermediate part is formed between the puncturing needle part and the drug introducing needle part and connects them smoothly by changing the diameter continuously.

Traditionally such cannula's are manufactured by forging by means of reciprocating tools applying force to the outer surface of the cannula. One example of such a manufacturing process may be seen in U.S. 2005/0015062 A1, which discloses a narrowing device having dies holding a needle pipe while rotating. The dies successively compress the needle pipe while impacting the needle pipe reciprocating diametrically. Thus, a narrowing step is performed.

Further background art may be seen in EP 0 995 453, DE 12 42 795, EP 0 271 775, U.S. Pat. No. 3,906,932 and U.S. Pat. No. 3,540,112.

Microscopy of cross-sections of needles manufactured by means of the above method has revealed that the method yields needles with asymmetrical inner and/or outer surface. Asymmetrical outer surfaces result in an increased level of pain, as the needle in one direction transverse to its longitudinal direction, is larger than necessary. Asymmetrical inner surfaces result in increased flow resistance.

Accordingly, it is an object of a preferred embodiment of the present invention to provide a method of manufacturing a needle with reduced diameter, and no or little asymmetry of the inner and outer surface of the needle.

Additionally, it is an object of a preferred embodiment of the present invention to provide a method of manufacturing wherein the symmetry of the needle is not dependent on exact alignment of tool parts.

Furthermore, it is an object of a preferred embodiment of the present invention to provide a method of manufacturing which yields needles with low variation in the symmetry of the outer and inner surface.

Additionally, it is an object of a preferred embodiment of the present invention to provide a method wherein the stress on the tool parts is reduced.

### BRIEF DESCRIPTION OF THE INVENTION

In a FIRST aspect the present invention relates to a method for manufacturing a cannula for an injection device, the cannula defining a tapered end part terminating in a distal end for insertion into the tissue of a living being, the method comprising the steps of:
providing a tubular element which defines a distal end, a proximal end and a conduit extending between the distal end and the proximal end,
providing a tool defining a tapered cavity,
reducing the hardness of a distal zone of the tubular element, the distal zone extending from the distal end towards the proximal end, and subsequently
forcing the distal end of the tubular element into the tapered cavity so as to define a tapered end part of the tubular element, said tapered part defining the distal end.

Advancing the distal end of the annular/tubular element into the tapered cavity of the tool, forces the distal end to be reshaped by plastic forming such that the outer shape of the annular/tubular element corresponds to the inner shape of the tool. Accordingly, a tapered zone is defined on the annular/tubular element.

One advantage of this method is that by using a tool which does not comprise two reciprocating tool parts, the risk of manufacturing cannula's with asymmetrical shape is reduced or even eliminated. Moreover, guiding means for ensuring alignment of reciprocating tool parts may be dispensed with whereby a simpler manufacturing process is provided. Furthermore, as the position of the tool is identical for each manufacture of a cannula, the variation of the outer dimensions of the cannula's is reduced considerably.

In the context of the present invention the term “cannula” shall be understood as an annular/tubular element suitable for introducing a medicament into the skin, subcutaneous tissue, a muscle, a blood vessel, or body cavity of a living being.

The cannula may comprise a metal material such as one or more of the following metal materials: nickel, titanium, stainless steel, and noble metals such as silver, gold, palladium, rhenium, and iridium. The stainless steel metal may comprise chromium and nickel, such as 19% chromium and 9% nickel. Other types of stainless steels than the mentioned austenitic grade may be used, such as ferritic, duplex, martensitic or precipitation hardening stainless steel. The cannula may comprise any alloy comprising any of the aforementioned metal materials.

Furthermore, in the context of the present invention the term “injection device” shall be understood as any stationary or portable device adapted to house a medicament and to eject said medicament into a living being. Examples of injection devices are—but are not limited to—syringe devices, injection pens and pumps for continuous delivery of the medicament.

In one embodiment the injection device comprises a compartment for accommodation of a medicament, which compartment forms an integral part of the injection device. In other embodiments the compartment is exchangeable such that when a first compartment is empty a second compartment may be inserted into the device.

Additionally in the context of the present invention, the term “tapered part” shall be understood as a part wherein the diameter gradually decreases or increases. The tapered part may be convex, concave or define a cone.

Similarly in the context of the present invention, the term “tapered cavity” shall be understood as any cavity wherein the diameter gradually decreases towards a bottom of the cavity.

The distal part forms the needle point or a cutting edge which is sharp enough to penetrate tissue of a living being. In one embodiment this means that the force required to penetrate the skin with the needle point or cutting edge is below 1 N.

The tool which comprises a tool member defining a tapered cavity may comprise any feature or element of the second aspect of the invention. Accordingly, the cavity may define a shape forming zone for shaping the tubular element when advanced into said zone, and a guiding zone for guiding an tubular element into the shape forming zone.

The method comprises the step of reducing the hardness of a distal zone of the tubular element, the distal zone extending from the distal end towards the proximal end. The step of reducing the hardness precedes the step of forcing, whereby the force needed to reshape the tubular element is reduced. Furthermore, tear and wear of the tools is reduced.

In one embodiment the hardness of the entire tubular element is reduced while in other embodiments only that part of the tubular element which will be reshaped is softened. This provides the advantage that the remaining parts of the cannula—i.e. those parts which are not to be reshaped—remains hard, and, thus, do not bend when the tubular element is reshaped.

The step of reducing the hardness may comprise the step of elevating the temperature of the distal zone. The temperature may be elevated by application of fire and/or high current and/or heating of the tool and/or by directing a laser beam towards the distal zone.

Moreover, the method may comprise the step of subjecting the tool to ultrasonic vibrations during which the distal end of the tubular element is forced in the distal direction and reshaped. During such ultrasonic agitation the opening of the cavity of the tool changes between a larger and a smaller diameter. When the cavity defines the larger diameter the tubular element is allowed to be advanced into further into the cavity and when the cavity defines the smaller diameter the tubular element is deformed plastically. The tool may be agitated radially or parallel with a centre axis defined by the cavity.

In the context of the present invention the term “ultrasonic” shall be understood as mechanical vibrations at a frequency above 10 kHz.

The method may comprise the step of forming a flow passage in the distal zone, the flow passage extending between an outer surface of the tubular element and an inner surface defined by the tubular element, the inner surface being a sidewall defining a conduit of the tubular element.

The invention according to the first aspect may comprise any combination of features and/or elements of the second and/or third aspect of the invention. As an example the cannula may be made by means of a monolithic tool according to the second aspect of the invention.

In a SECOND aspect, the present invention relates to a tool for providing a tapered portion of a cannula for an injection device, the tool defining a tapered cavity. The tool may define a surface from which the cavity extends into the tool.

The cavity may define a shape-forming zone for shaping a work piece advanced into said zone, and a guiding zone for guiding a work piece into the shape forming zone. An angel defined between the sidewall of the shape-forming zone and a centre axis of the cavity may be below 30 degrees, such as below 20 degrees, such as below 15 degrees. An angle defined between a sidewall of the guiding zone and a centre axis of the cavity may be in the range of 15-60 degrees, such as in the range of 30-45 degrees. Preferably, the guiding zone and/or the shape-forming zone have smooth surfaces i.e. having a roughness Rz below 10 μm, such as below 2.5 μm.

The guiding zone may extend between the surface of the tool and the shape forming zone. Accordingly, an tubular element advanced into the tapered cavity and striking the guiding zone will be guided towards the shape-forming zone when advanced further into the tapered cavity.

The widest internal diameter of the shape-forming zone may be in the range of 0.3-2 mm, such as 0.35-0.5 mm. The narrowest diameter of the shape-forming zone may be in the range of 0-0.3 mm, such as 0.1-0.25, such as 0.1-0.2 mm.

In order to avoid moving tool parts, the tool may be monolithic i.e. fabricated as a single structure and without seams. Alternatively, the tool may be defined by two tool parts which are permanently fixed to each other and/or non-suitable-for being moved relative to each other during shaping of the tubular element.

The invention according to the second aspect may comprise any combination of features and/or elements of the first and/or third aspect of the invention.

In a THIRD aspect, the present invention relates to a cannula for an injection device, the cannula defining a tapered end part terminating in a distal end for insertion into the tissue of a living being, the cannula defining a first zone wherein the hardness of the cannula decreases in the direction of the distal end.

Normally when manufacturing a tapered cannula, the tapered part is harder than the non-tapered part. As harder parts are more prone to breakage than less hard parts, the result is a cannula having a tendency of breaking in a zone which during use is inserted into the tissue of the user. Breakage inside the user is highly undesirable as it is difficult if not impossible to remove the broken part from the body without surgery. The present invention utilises that softer parts are less prone to breakage, as such softer parts may be bent a plurality of times prior to breakage, relative to harder parts. In one embodiment, the first zone is defined in the tapered part (the tissue penetrating part). Accordingly, the tissue penetrating part is less prone to breakage, whereby breakage inside the patient less likely.

Furthermore, the cannula may comprise a second zone defined between the first zone and the distal end, the hardness of the cannula in said second zone increasing in the direction of the distal end. The second zone may be provided in the tapered end part and/or in the non-tapered end part.

By providing a tip (the second zone) which is harder than the first zone, the tip point itself is less prone to bending. Thus, any radial force applied to the tip results. in bending in the first zone, whereby a hard tip may be combined with prevention of breakage inside the patient.

It will be appreciated that in order for a user to bend the first zone back to its original shape, it must be possible for the user to get a firm grip of the cannula on both sides of the bent zone. The closer the first zone is to the tip the more difficult it is to force the first zone back to its original shape. Thus, by providing the first zone close to the tip, the user cannot reshape a bend first zone and will dispose a bend cannula. This is desirable, as cannula's which break inside the patient often are cannula's which have been reshaped by the user.

Moreover the harder the tip is, the more difficult it is to bend the tip itself. As even the smallest bending of the tip increases the pain experienced by the user, it is desirable that such bending is avoided.

At least a part of the first zone may be defined in the tapered end part. In one embodiment the hardness of the cannula in the distal end is lower than the hardness in an opposite proximal end of the cannula. In another embodiment, the hardness of the cannula in the distal end is lower than in any non-tapered part of cannula. Moreover, the hardness of the cannula in any non-tapered part may be lower than in the hardness in the distal end.

The distal end of the tapered cannula may define a cutting edge or a needle point.

In one embodiment, the cannula defines an attachment zone which is attached to a connecting element. The connecting element may be adapted to secure the cannula to an injection device. The connecting element may be threaded such that the connection element may be screwed onto a syringe device comprising a corresponding thread. The attachment zone and the first zone may coincide. Alternatively, the first zone may be defined between the attachment zone and the distal end.

The cannula may have an overall length in the range of 4-30 mm, such as in the range of 10-25 mm, such as in the range of 18-22 mm, such as approximately 20 mm.

The length of the non-tapered part of the cannula may be in the range of 5-25 mm, such as in the range of 10-20 mm, such as 15 mm.

The length of the tapered part of the cannula may be in the range of 1-25 mm, such as in the range of 5-20 mm, such as in the range of 10-15 mm.

The outer diameter of the cannula may be in the range of 0.1-0.5 mm, such in the range of 0.2-0.4 mm, such as 0.3 mm.

The inner diameter of the cannula may be in the range of 0.05-0.4 mm, such as in the range of 0.1-0.3 mm, such as 0.2 mm.

The diameter of the narrowest part of the tapered portion may be in the range of 10-90% of the diameter of the widest part of the tapered portion, such as in the range of 20-80%, such as in the range of 40-60%.

The cannula may comprise an tubular element defining a conduit extending between the proximal end and the distal end. A flow passage may be defined in a sidewall of the tubular element so as to provide fluid connection between an outer surface of the cannula and the conduit. The diameter of the flow passage may be in the range of 0.01-0.20 mm.

The hardness of the cannula may be in the range of 150-70O HV. In one embodiment the first zone has a hardness in the range of 150-400 HV, such as in the range of 150-200 HV, and the second zone has a hardness in the range of 200-700 HV, such as in the rang of 300-400 HV.

The invention according to the third aspect may comprise any combination of features and/or elements of the first and/or second aspect of the invention. As an example the cannula may be made by means of a monolithic tool according to the second aspect of the invention.

### DETAILED DESCRIPTION OF THE INVENTION

The invention will now be described in further detail with reference to the drawings in which:

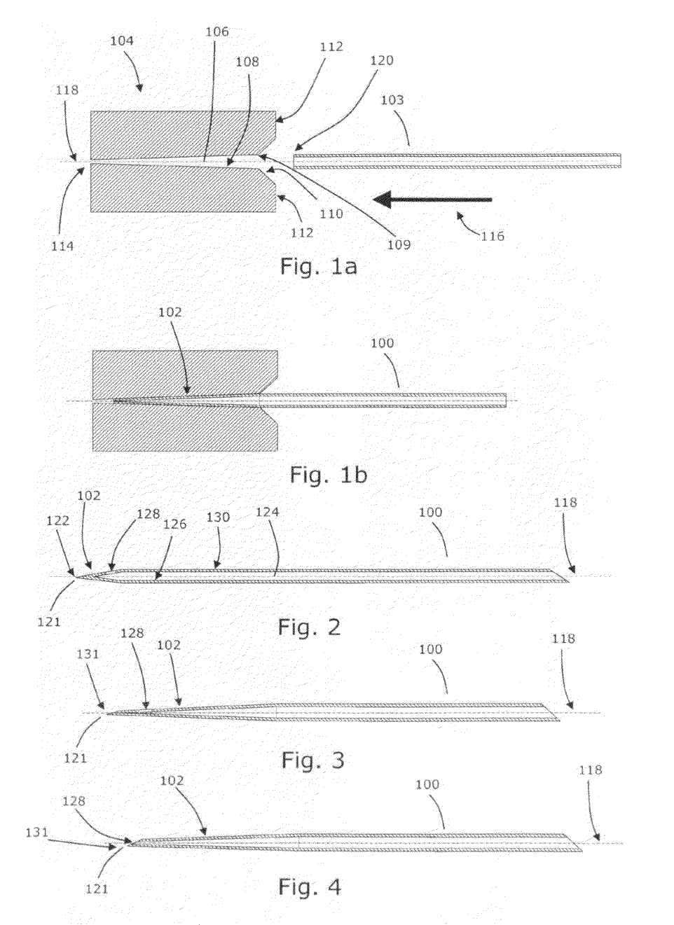

FIGS. 1a-b disclose steps in the method according the first aspect of the invention,

FIGS. 2-4 disclose tapered cannula's according to the third aspect of the invention, and

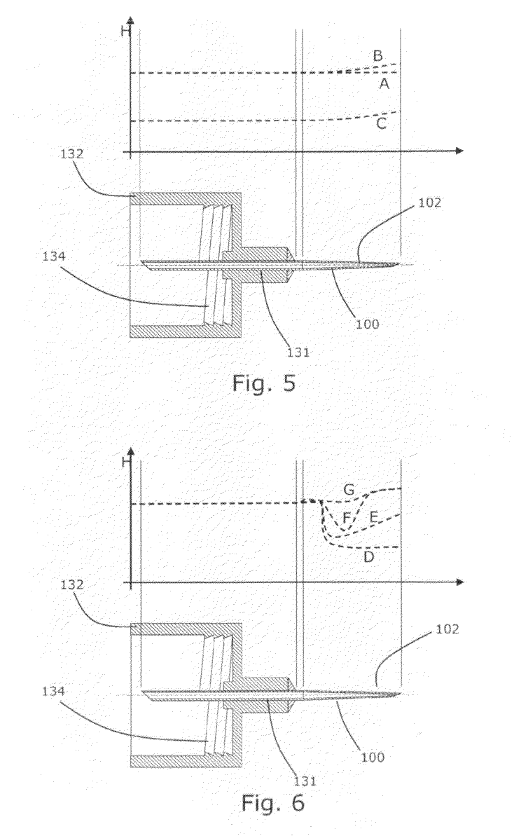

FIGS. 5-6 disclose the hardness of cannula's annording to the third aspect of the invention.

FIGS. 1a and 1b disclose steps in the method for manufacturing a cannula 100 with a tapered end portion 102. Initially an tubular element 103 and a tool 104 defining a tapered cavity 106 are provided. The cavity 106 defines a shape-forming zone 108 and a guiding zone 110 which extends between an outer surface 112 and the shape-forming zone 108. In a transition 109 between the shape-forming zone 108 and the guiding zone 110 the diameter of the two zones are substantially identical. The diameter of the shape-forming zone 108 decreases from the transition 109 towards a bottom part 114 of the cavity i.e. in the left direction in the drawing. The diameter of the guiding zone 110 increases from the transition 109 towards the outer surface 112, i.e. in the right direction in the drawing. In order to form the tapered end portion 102, the tubular element 103 is forced into the tapered cavity as indicated by arrow 116. If the tubular element is not exactly aligned with the tool 104, i.e. if their centre axes 118 do not coincide, the guiding zone 110 guides the tubular element 103 into.the shape-forming zone 108. When the distal end 120 of the tubular element 103 reaches the transition 109, the force needed to advance the tubular element further into the tapered cavity 106 increases. By advancing the tubular element 103 further into the shape-forming zone 108, the tubular element 103 is reshaped by plastic forming a distal end part of the tubular element, and the cannula 100 is formed.

While advancing the tubular element 103 into the shape-forming zone 108, the tool 104 may be subjected to ultrasonic vibrations.

FIGS. 2-4 disclose different embodiments of cannula's 100 with tapered end portions 102. The diameter of the cannula's 100 in the area of the tapered end portions decrease in the direction of the distal end 121 of the cannula.

In FIG. 2 the cannula 100 defines a needle point 122 for penetrating the skin of a patient. Moreover, the cannula defines a conduit 124 extending in the longitudinal direction of the cannula. The conduit is encircled by a sidewall 126 of the cannula. A flow passage 128 is defined in the tapered end part 102. The flow passage 128 which provides a flow connection between an outer surface 130 of the cannula and the conduit extends between the outer surface 130 and the sidewall 126.

In FIG. 3 the cannula 100 comprises a cutting edge 131 which is provided by cutting/grinding the distal end in a direction transverse to the longitudinal axis 118 of the cannula. The cut/grind is provided in a zone wherein the sidewalls of the tubular element abut (is plastically joint together) whereby no flow passage is defined in the distal most end of the device. Thus, in order to allow fluid to pass from the conduit to the outer surface, a flow passage 128 is defined in tapered portion 102.

In FIG. 4 the flow passage 128 is provided by cutting/grinding the distal end in a direction transverse to the longitudinal axis of the cannula. The cut/grind is provided in a zone wherein the sidewalls of the cannula define a conduit i.e. they do not abut each other. Thus, a flow passage 128 is defined in the distal most end of the device.

FIG. 5 discloses a cannula 100 having a tapered end portion 102. The cannula 100 defines an attachment zone 131 which is attached to a connecting element 132. The connecting element 132 comprises a threaded portion 134 for securing the connecting element to a syringe device. The line A represents the hardness of a cannula with uniform hardness. The line B represents a cannula subjected to deformation hardening at the distal portion. The line C represents a cannula subjected to overall annealing before applying deformation hardening of the distal portion.

In FIG. 6 the hardness of the cannula has been reduced in the area of the tapered end part, prior to its shaping. Each of the lines indicates the hardness of the cannula along its length. The line D illustrates a cannula wherein the proximal end is harder than the distal end. The tapered end part comprises a first zone (i.e. wherein the hardness decreases in the distal direction) and the cannula comprises no second zone (i.e. a zone wherein the hardness increases in the distal direction). In the examples of the lines E, F and G, the tapered end part comprises a second zone positioned between the first zone and the distal end of the cannula. Line D represents a cannula subjected to local annealing at the distal portion. Line E and F represent cannula's subjected to local annealing and deformation hardening at the distal end portion. Line G represents a cannula subjected to deformation hardening at the distal end portion.

1. A method for manufacturing a cannula for an injection device, the cannula defining a tapered end part terminating in a distal end for insertion into the tissue of a living being, the method comprising:
providing a tubular element which defines a distal end, a proximal end and a conduit extending between the distal end and the proximal end,
providing a tool defining a tapered cavity,
reducing the hardness of a distal zone of the tubular element, the distal zone extending from the distal end towards the proximal end, and subsequently
forcing the distal end of the tubular element into the tapered cavity so as to define a tapered end part of the tubular element, said tapered part defining the distal end.

2. A method according to claim 1, wherein the reducing the hardness further comprises elevating the temperature of the distal zone.

3. A method according to claim 2, wherein the elevating the temperature further comprises elevating the temperature by directing a laser beam towards the distal zone.

4. A method according to claim1, further comprising subjecting the tool member to ultrasonic vibrations.

5. A method according to claim 1, further comprising forming a flow passage in the distal zone, said flow passage extending between an outer surface of the tubular element and an inner surface defined by the tubular element.

6. A tool for providing a tapered portion of a cannula for an injection device, the tool defining a tapered cavity.

7. A tool according to claim 6, wherein the tool defines a surface and wherein the cavity extends from the surface and into the tool.

8. A tool according to claim 6, wherein the cavity defines a shape-forming zone for shaping a work piece advanced into said zone, and a guiding zone for guiding a work piece into the shape forming zone.

9. A tool according to claim 8, wherein the guiding zone extends between the surface and the shape-forming zone.

10. A tool according to claim 6, wherein the widest internal diameter of the shape-forming zone is in the range of 2-4 mm.

11. A tool according to claim 6, wherein the narrowest diameter of the shape-forming zone is in the range of 0.01 and 0.02 mm.

12. A tool according to claim 6, wherein the tool is monolithic.

13. A cannula for an injection device comprising:
a tapered end part terminating in a distal end for insertion into the tissue of a living being, and
a first zone wherein the hardness of the cannula decreases in the direction of the distal end.

14. A cannula according to claim 13, further comprising a second zone defined between the first zone and the distal end, and wherein the hardness of the cannula in the second zone increases in the direction of the distal end.

15. A cannula according to claim 14, wherein at least a part of the second zone is defined in the tapered end part.

16. A cannula according to claim 13, wherein at least a part of the first zone is defined in the tapered end part.

17. A cannula according to claim 13, wherein the hardness of the cannula in the distal end is lower than the hardness in an opposite proximal end of the cannula.

18. A cannula according to claim 13, wherein the hardness of the cannula in the distal end is lower than in any non-tapered part of cannula.

19. A cannula according to claim 13, wherein the hardness of the cannula in any non-tapered part is lower than in the hardness in the distal end.

20. A cannula according to claim 13, wherein the distal end defines a cutting edge.

21. A cannula according to claim 13, wherein the cannula defines an attachment zone which is attached to a connecting element, which is adapted to secure the cannula to an injection device.

22. A cannula according to claim 21, wherein the first zone is defined between the attachment zone and the distal end.

23. A cannula according to claim 13, wherein the cannula comprises a tubular element defining a conduit extending between the proximal end and the distal end and wherein a flow passage is defined in a sidewall of the tubular element so as to provide fluid connection between an outer surface of the cannula and the conduit.
